(19) United States
(12) Patent Application Publication
(10) Pub. No.: US 2008/0051654 A1
(43) Pub. Date: Feb. 28, 2008

(54) MEDICAL IMAGING APPARATUS, AN ULTRASONIC IMAGING APPARATUS, A VIEWER, AND A METHOD FOR RECORDING ULTRASONIC IMAGES

(75) Inventors: Ryota OSUMI, Nasushiobara-shi (JP); Takeshi Sato, Nasushiobara-shi (JP)

Correspondence Address: OBLON, SPIVAK, MCCLELLAND MAIER & NEUSTADT, P.C., 1940 DUKE STREET, ALEXANDRIA, VA, 22314, US

(73) Assignees: Kabushiki Kaisha Toshiba, Tokyo (JP); TOSHIBA MEDICAL SYSTEMS CORPORATION, Otawara-shi (JP)

(21) Appl. No.: 11/846,022

(22) Filed: Aug. 28, 2007

(30) Foreign Application Priority Data
Aug. 28, 2006 (JP) 2006-230133

(51) Int. Cl. A61B 8/00

(52) U.S. Cl. 600/437

(57) ABSTRACT

An ultrasonic imaging apparatus acquires image data that shows ultrasonic images from an image-acquisition unit. The acquired image data becomes video data combined with screen data in a video-data-generating unit. A record-control unit sends to a superposition unit the video data combined with the image data. The superposition unit is configured to superpose the image data or the screen data with information on the examination conditions. Once the information on the examination conditions is superposed by the superposition unit, the video data including the image data and the screen data is outputted to a recording device.

### BACKGROUND OF THE INVENTION

1. Field of the Invention

The present invention relates to the art of recording a medical image generated by a medical imaging apparatus, and the art of displaying the recorded medical image. In particular, it relates to the art of recording and displaying an ultrasonic image generated by sending/receiving ultrasound in an ultrasonic imaging apparatus.

2. Description of the Related Art

A medical imaging apparatus is an apparatus that is capable of acquiring information regarding body tissues of a subject to be examined. The medical imaging apparatus is also an apparatus that is capable of generating a medical image, based on the acquired information regarding the body tissues, showing the body tissues of the subject to be examined. Additionally, the medical image generated by the medical imaging apparatus can be also displayed on a viewer. A physician can view the medical image displayed on the viewer and examine/diagnose diseases of the subject to be examined. Additionally, the medical image generated by the medical imaging apparatus can be a still image or a moving image.

This medical imaging apparatus has various modalities. Examples of the medical imaging apparatus include an ultrasonic imaging apparatus, an X-ray imaging apparatus, an X-ray CT (Computed Tomography) apparatus, and a MRI (Magnetic Resonance Imaging) apparatus.

For example, the ultrasonic imaging apparatus acquires biological information of the subject to be examined by scanning the subject using ultrasound. The ultrasonic imaging apparatus also generates, from acquired biological information, tomographic images and other ultrasonic images such as movement and blood flow.

This ultrasonic imaging apparatus is capable of suppressing invasions (external stimulation that may disturb vivo homeostasis) accompanied by the acquisition of biological information of the subject to be examined.

Furthermore, the ultrasonic imaging apparatus is capable of displaying in real time the body tissues of the subject to be examined. The method for this real-time displaying of the body tissues of the subject is executed by the B-mode method and the Doppler method. In other words, the ultrasonic imaging apparatus overlaps and displays the ultrasonic (tomographic) images acquired by user's operation in a time sequence (i.e. in order of acquisition), so as to display in real time the body tissues and blood flows of the subject. Alternatively, the ultrasonic imaging apparatus displays in real time the body tissues and blood flows of the subject by displaying a plurality of tomographic imaging arranged in a time sequence.

However, the ultrasonic images acquired by this ultrasonic imaging apparatus are not only displayed and viewed in real time. The ultrasonic images can be recorded in a recordable media for later viewing. The ultrasonic imaging apparatus outputs the ultrasonic image data to a recording device in order to record ultrasonic-image data in the recordable media.

As described above, in some cases, the ultrasonic image data that is outputted to the recording device may need supplemental information such as information regarding the examination conditions. This means information such as the imaging conditions that are necessary for reproducing the ultrasonic image based on the recorded ultrasound image data. Furthermore, information on the examination conditions includes, for example, information about the subject that is necessary for viewers of the images to re-measure the subject to be examined when the ultrasonic images are reproduced.

Examples of information on the examination conditions also include information on the operating conditions of the ultrasonic imaging apparatus, such as display magnification, sweep speed, and returning speed. Additionally, other examples include information on the measurements of the subject to be examined (synchronization information on an electrocardiogram).

While viewing the ultrasonic images that are displayed in real time, operators work to acquire biological information through the ultrasonic imaging apparatus. In this case, the relevant operators can refer to the information on the examination conditions right then and there. For example, because operators set the display magnification according to the situation in which they are in, the operators can immediately refer to this information during operation. Additionally, operators can also refer to the electrocardiogram of the subject to be examined when necessary.

In other cases, viewers of the recorded ultrasonic images might only view the ultrasonic images without seeing the information on the examination conditions at the time of the acquisition operation. For example, there is a possibility that the operators (e.g. engineers) of the ultrasonic imaging apparatus might not be the viewers (e.g. physicians) of the ultrasonic image. Even if both are the same person, there may be a wide range of information on the examination conditions, making it difficult for the viewers of the ultrasonic image to remember all the information on the examination conditions until the ultrasonic images are reproduced.

Accordingly, when recording ultrasonic images, it is necessary that the ultrasonic imaging apparatus is comprised to record the ultrasonic images to be recorded as well as the information on the examination conditions. However, some information on the examination conditions may change over time in accordance with the ultrasonic images. For example, the synchronization information from an electrocardiograph at the time of examination keeps changing.

In other words, the ultrasonic imaging apparatus collects images in synchronization with the heartbeat of the subject. The ultrasonic imaging apparatus collects images at a specified time after the electrocardiogram's R-wave is detected, and in this case, the images are collected by 1 frame per heartbeat throughout a plurality of heartbeats.

This synchronization information of the electrocardiogram is used for observing the cardiac motions in a single time phase through a plurality of heartbeats, and changes over time in accordance with the ultrasonic images. Therefore, it is necessary that the ultrasonic imaging apparatus links the synchronization information from the electrocardiogram with the changes over time that occur over time in the ultrasonic images, and records it together with the ultrasonic images. In this way, if the changes over time of the ultrasonic images are linked with the synchronization information from the electrocardiogram, a device for reproducing ultrasonic images can continuously reproduce by reading out the recorded ultrasonic images as well as the synchronization information from the electrocardiogram.

Furthermore, it is possible that other information on the examination conditions, such as display magnification, sweep speed, and operating conditions of an ultrasonic probe, may change depending on the status of usage. Accordingly, such information on the examination conditions also changes over time in accordance with the ultrasonic images, and it is necessary to link it with ultrasonic images for recording.

Conventionally, this ultrasonic image data has been recorded in a videocassette tape using an analog VCR (Video Cassette Recorder). However, digital video recording standards, such as a DVD (Digital Versatile Disc), have been prevalent in recent years. Owning to the diffusion of these digital video recording standards, more and more ultrasonic image data is being recorded in recordable media based on digital video recording standards such as DVD instead of VCR.

The recording device records the ultrasonic-image data generated by the ultrasonic imaging apparatus in accordance with the data format of the relevant recording device and recordable media. In other words, some recording devices record the ultrasonic-image data under various digital video recording standards, and other recording devices record it under various analog video recording standards. Thus, the ultrasonic imaging apparatus also has to output the ultrasonic image data in accordance with the data format (video recording standards) of the recording device.

For the above purpose, an invention is disclosed that, when an ultrasonic imaging apparatus is used to input images from an image-inputting unit into an image-recording unit, an operator sets an image format for each individual image-inputting unit, selecting automatically the most appropriate image format from the image-recording unit of the ultrasonic imaging apparatus to each device (e.g. Japanese Unexamined Patent Application Publication H8-154244).

The storage area in the recordable media for recording this information regarding the examination conditions differs depending on the data format of the recordable media. Analog VCRs recorded (added) information regarding the examination conditions in an area other than the area from which images are displayed out of the video signal, in an area otherwise known as VBI (Vertical Blanking Interval). By doing so, in respect to the changes made to ultrasonic imaging apparatuses over time, an analog VCR can record the information regarding the examination conditions while adapting to the changes over time made over time to ultrasonic images.

On the other hand, a recording device using digital video recording standards, such as a DV (Digital Video) digital VCR, is not capable of recording the information regarding the examination conditions in a manner that responds to each frame constituting the video data. Therefore, recording devices under digital video recording standards have recorded the information on the examination conditions in a storage area for recording data other than video data. Additionally, some medical institutions may use several different types of recording devices due to different data formats. In line with this fact, some medical institutions may use several different types of reproducing devices and viewers for the ultrasonic images due to different data formats.

Accordingly, it is preferable that the data recorded by different data formats, such as ultrasonic image data recorded under analog recording standards and ultrasonic image data recorded under digital video recording standards, can be recorded and reproduced on either of the devices.

However, in conventional methods for adding information regarding the examination conditions, differences in image data formats have resulted in different recording methods, as described above. Therefore, between the different formats, the recorded information on the examination conditions could not be used indiscriminately because of the differences in the recording areas and the recording formats, even when the image data itself could be used indiscriminately between different formats.

For example, some medical institutions may connect the VCR with a DVD drive to output the ultrasonic image data from the VCR to the DVD drive, so as to reproduce and also record ultrasonic images that have been recorded in a VCR. In this case, the VCR can output the image data itself to the DVD drive, but there is still the possibility that the information on the examination conditions that has been recorded in the VCR is not available due to the difference in the formats.

As described above, the information on the examination conditions is not available indiscriminately between different formats, which means that the viewers of the recorded ultrasonic images cannot recognize the information necessary for diagnosis and re-measurement of the subject. Consequently, the reproducing device and the recording device of the ultrasonic images cannot mutually use ultrasonic images that have been recorded in different formats.

This will lead to a problem not only between the analog video recording standards and the digital video recording standards, but also within the digital video recording standards. For example, in some cases, the devices under the digital video recording standards are connected and the ultrasonic image data is outputted from one device to the other device. In this case, the data transfer will often be performed using an analog method, even between devices under the digital video recording standards. Since the information on the examination conditions is recorded in the recording area using the digital video recording standards, the analog data transfer creates the possibility that the information on the examination conditions itself will not be transferred.

Additionally, in some cases, devices with different data formats under digital video recording standards are connected and the ultrasonic image data recorded in one data format is outputted to a device with the other format. In this case, even if the data transfer is performed using a digital method, the information on the examination conditions cannot be identified due to the different data format when reproducing the ultrasound images. Therefore, there is a possibility that the ultrasound images cannot be mutually used between the different formats.

Furthermore, in the devices under digital video recording standards, different recording areas are used for information displaying the video and other information. In other words, information other than the information displaying the video cannot be added onto the information showing the video data when recorded in the recording area for the video data. In particular, the information on the examination conditions is classified in the information other than information displaying the video. Therefore, the information on the examination conditions is to be recorded in, out of the entire recording area, a data recording area other than the data recording area for the ultrasonic images. As a result, the information on the examination conditions and the video data are each separated for recording. Thus, there is a possibility that the recording of the information on the examination conditions will not be performed in response to the changes over time of the ultrasonic images.

As described above, the information on the examination conditions may not be used due to the difference in the data formats. This difference may also prevent the recording of the information on the examination conditions in response to the changes over time of the ultrasonic images. These problems make it difficult to seize the information on the examination conditions that responds to the changes over time of the ultrasonic images, in cases where the viewers diagnose the subject by viewing the ultrasonic images. Furthermore, the viewers cannot recognize the necessary information, which will lead to the possibility of causing interference for diagnoses.

An image-recording device according to Japanese Unexamined Patent Application Publication H8-154244 is capable of setting a data format for every image-inputting unit when the operator records image data. Therefore, this image-recording device is capable of automatically selecting the most appropriate data format when outputting the image data. However, the difficulty of mutually using the ultrasonic images, which is a problem caused by the difference in the data formats between different formats, as well as the problems in the responsiveness of the information on the examination conditions to the image data that changes over time, have not been solved.

### SUMMARY OF THE INVENTION

The present invention is directed to provide art that allows for the use of information on the examination conditions when recorded medical image data is reproduced and recorded mutually between devices with different data formats. Additionally, the present invention is also directed to provide art that enables the information on the examination conditions to be recorded in response to the changes over time of the medical images.

The first embodiment of this invention is an ultrasonic imaging apparatus, which acquires the information on the interior of a subject to be examined by radiating ultrasound to the subject and generates image data showing the internal conditions of said subject based on the information on the interior of said subject, comprising: a video-data-generating unit configured to generate video data to be displayed on a display screen, based on said image data and screen data to display an image display area different from an area in which the image based on said image data is displayed; and a superposition unit configured to superpose information on the examination conditions on the image data used for generating said video data, wherein said video data, including the image data superposed with said information on the examination conditions by said superposition means, is outputted to a recording device.

Additionally, the second embodiment of the present invention is an ultrasonic imaging apparatus, which acquires the information on the interior of a subject to be examined by radiating ultrasound to the subject and generates image data showing the internal conditions of said subject to be examined based on the information on the interior of said subject to be examined, comprising: a video-data-generating unit configured to generate video data to be displayed on a display screen based on said image data and screen data to display an image display area different from an area in which the image based on said image data is displayed; and a superposition unit configured to superpose information on the examination conditions onto data to display characters, figures, marks and patterns that do not change over time out of said screen data used for reproducing said video data; wherein said video data, including the screen data superposed with said information on the examination conditions by said superposition means, is outputted to a recording device.

According to the first embodiment and the second embodiment of the present invention, the ultrasonic imaging apparatus is configured to superpose the information on the examination conditions obtained when collecting information on the inner tissues of the subject onto the image data showing the inner tissues of the subject or the data showing characters/marks that do not change over time. Therefore, the area for recording the information on the examination conditions can be shared even between different data formats.

As a result, in the case of recording ultrasonic images in certain recordable media and reproducing it in a data format different from the data format related to that recordable media, the information on the examination conditions can be extracted and recognized, and therefore the recorded ultrasonic images can be used interchangeably between different data formats (e.g. between analog video recording standards and digital video recording standards).

Even in cases of recording the ultrasonic images by using digital video recording standards, since the information on the examination conditions is superposed on the video data itself, the superposed information on the examination conditions can be recognized and the information on the examination conditions that changes over time can be recognized at the time of reproducing said ultrasound diagnostic images. Therefore, in the digital video recording standards, the information on the examination conditions can be recognized in response to the reproduction of the ultrasonic imaging apparatus that changes over time. Additionally, it can be displayed in an indistinctive form at the time of the observation of the image, thus resulting in no possibility of interference in the observation of the relevant images.

It should be noted that the present invention can be applied not only to an ultrasonic imaging apparatus but also a medical imaging apparatus according to other modalities.

### BRIEF DESCRIPTION OF THE DRAWINGS

FIG. 1 is a block diagram illustrating a schematic configuration of an ultrasonic imaging apparatus according to an embodiment of the present invention.

FIG. 2 is a schematic view illustrating a superposing area of supplemental information in an ultrasonic imaging apparatus according to the first embodiment of the present invention.

FIG. 3A is a schematic view of a display screen 300 illustrating each frame or specified-interval frame of image data.

FIG. 3B is a schematic partial enlarged view illustrating the conditions after an ultrasonic image display area 201 of a display screen 300 is divided to an image block 301.

FIG. 4A is a schematic view illustrating a part of the conditions before an image block 301 of an image data frame undergoes orthogonal transformation.

FIG. 4B is a schematic view illustrating the conditions of the process of superposing the supplemental information by performing an orthogonal transformation (e.g. a discrete cosine transform) on an image block 301 of image data, and decomposing to a spatial frequency in superposition unit 70

FIG. 5 is a flow chart illustrating a series of operations performed by an ultrasonic imaging apparatus 1 according to the first embodiment of the present invention.

FIG. 6 is a flow chart illustrating a series of operations performed by an ultrasonic imaging apparatus 1 according to the first embodiment of the present invention.

FIG. 7 is a figure illustrating a superposing area of supplemental information in an ultrasonic imaging apparatus 1 according to the second embodiment of the present invention.

FIG. 8 is a block diagram illustrating a schematic configuration of an ultrasonic imaging apparatus 1 according to a modified example of the present invention.

FIG. 9 is a block diagram illustrating a schematic configuration according to a modified example of the present invention.

### DETAILED DESCRIPTION OF THE EMBODIMENTS

### First Embodiment

Configuration

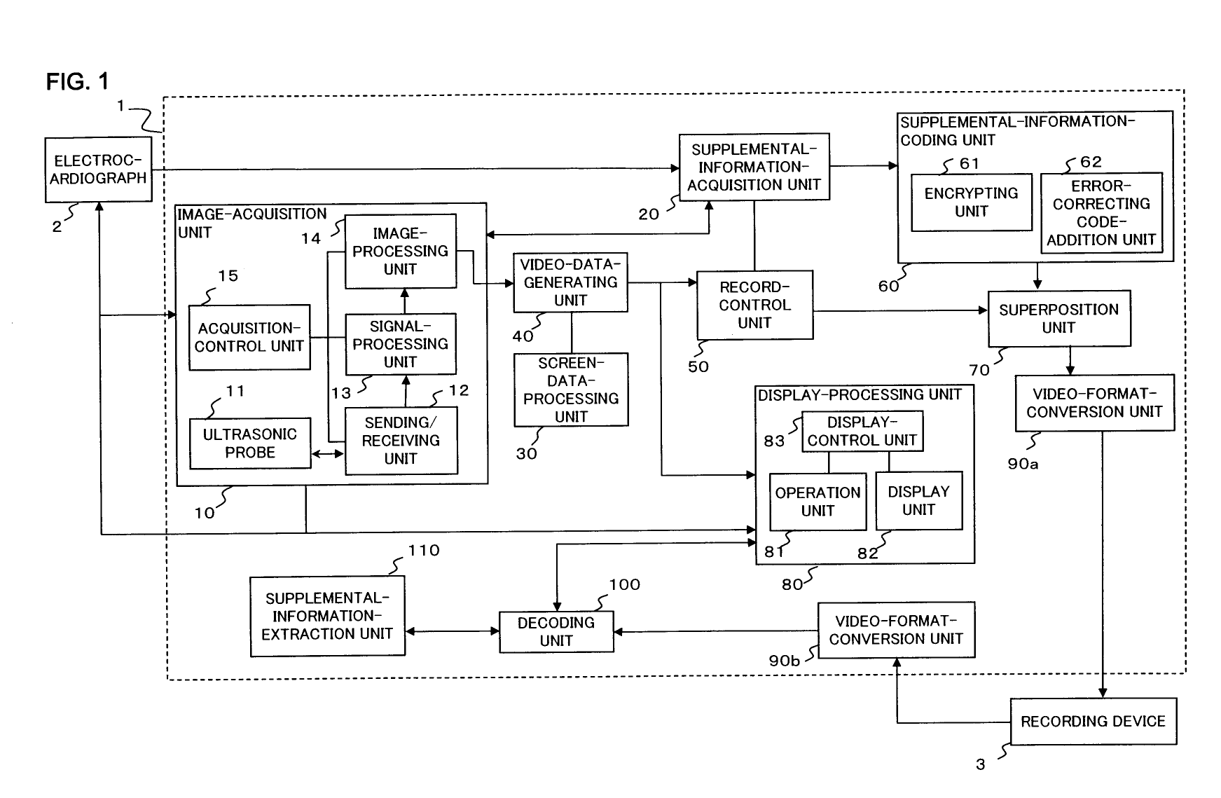

The configuration of an ultrasonic imaging apparatus 1 according to the first embodiment of the present invention will be described with reference to FIG. 1. FIG. 1 is a block diagram illustrating a schematic configuration of an ultrasonic imaging apparatus 1 according to an embodiment of the present invention.

The ultrasonic imaging apparatus 1 according to this embodiment is configured to comprise an image-acquisition unit 10, a supplemental-information-acquisition unit 20, a screen-data-processing unit 30, a video-data-generating unit 40, a record-control unit 50, a supplemental-information-coding unit 60, a superposition unit 70, a display-processing unit 80, a video-format-conversion unit 90a/90b, a decoding unit 100, and a supplemental-information-extraction unit 110.

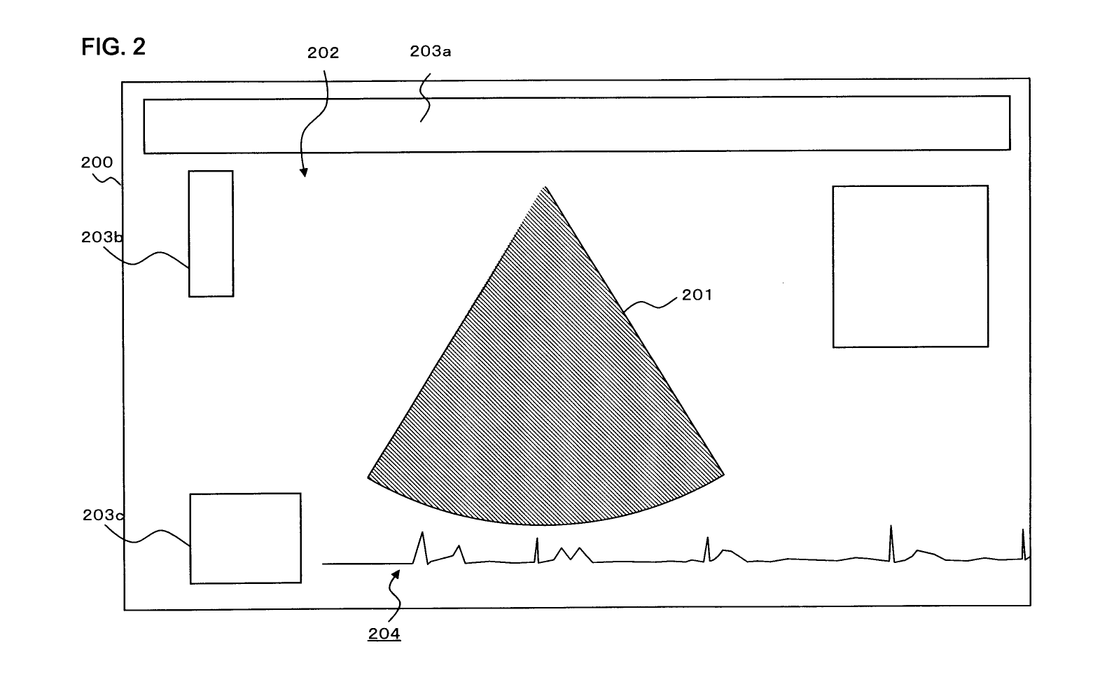

The ultrasonic imaging apparatus 1 features the superposition unit 70. The superposition unit 70 superposes the supplemental information onto the image data used for displaying the ultrasonic images showing the inside of the subject (201 shown in FIG. 2). Examples of this supplemental information include synchronization information from an electrocardiogram, information on the operating conditions for the ultrasonic imaging apparatus 1, and other information on the operating conditions for an ultrasonic probe 11. This superposition unit will be described later. The configuration of each unit for the ultrasonic imaging apparatus 1 will be described below.

The image-acquisition unit 10 is configured to comprise an ultrasonic probe 11, a sending/receiving unit 12, a signal-processing unit 13, an image-processing unit 14, and an acquisition-control unit 15. The sending/receiving unit 12 sends electric signals to the ultrasonic probe 11. The ultrasonic probe 11 receives this electric signal and converts it into an ultrasonic pulse. Operators transmit this converted ultrasonic pulse to the subject by operating the ultrasonic probe 11. This ultrasonic pulse reaches the body tissues of the subject and is reflected as an echo.

The ultrasonic probe 11 receives the echo from the body tissues of the subject and converts it into the electric signal. The ultrasonic probe 11 sends this echo signal to the sending/receiving unit 12. The sending/receiving unit 12 sends the received echo signal to the signal-processing unit 13. The signal-processing unit 13 performs signal processing for the echo signal received from the sending/receiving unit 12. The signal-processing unit 13 sends the relevant processed data to the image-processing unit 14 after performing the signal processing. The image-processing unit 14 receives the data from the signal-processing unit 13 and generates the image data showing the body tissues of the subject. The acquisition-control unit 15 entirely controls each unit in the image-acquisition unit 10.

The ultrasonic probe 11 is configured to be equipped with an acoustic matching layer, ultrasonic transducers, a backing material formed on the back of the ultrasonic transducer, electrodes formed on the front and back of the ultrasonic transducers, and a connection lead connecting the relevant electrodes and the sending/receiving unit 12, which are not shown in the figure. A one-dimensional array probe or a two-dimensional array probe is used for the ultrasonic probe 11. The one-dimensional array probe is configured so that the ultrasonic transducers are arranged in one line in the direction of scanning. The two-dimensional array probe is configured so that the ultrasonic transducers are two-dimensionally arranged, and acquires three-dimensional biological information by executing a volume scan.

The sending/receiving unit 12 comprises a sending unit that supplies the electronic signal to the ultrasonic probe 11 to generate the ultrasound. In other words, the sending unit is comprised of a rate-pulse generator, a sending-delay circuit, and a pulsar. The rate-pulse generator generates a rate pulse that determines the rate cycle transmitted to the inside of the subject and supplies it to the sending delay circuit. This sending delay circuit provides a delay time used to focus the ultrasonic pulse in the specified depth and a delay time used for deflection to send the ultrasonic pulse in the specified direction. Subsequently, the sending-delay circuit supplies this rate pulse to the pulsar.

This pulsar comprises drive circuits. The pulsar also drives each ultrasonic transducer by applying drive signals generated by each drive circuit to the ultrasonic transducers in the ultrasonic probe 11. As a result, the ultrasonic transducers transmit the ultrasonic pulse to the subject.

Additionally, the sending/receiving unit 12 comprises a receiving unit that receives the signal from the ultrasonic probe 11. This receiving unit is comprised of a pre-amplifier, an analog/digital converter (ADC), a beam former, and an adder. The pre-amplifier amplifies the minute electric signal converted by the ultrasonic transducer. The fundamental and harmonic components of the relevant amplified electric signal are converted into the digital signal by the analog/digital converter and subsequently sent to the beam former.

The beam former sequentially changes the delay time used for focusing to focus the ultrasonic reflected wave and the receiving directionality of the ultrasonic reflected wave. The beam former also provides the delay time used for deflection to scan the subject to the received signal that has been converted into the digital signal.

The adder performs a phasing addition (wherein the received signal acquired from the specified direction is added with the phase) of the output from the beam former.

The signal-processing unit 13 is configured to comprise a B-mode (brightness mode)-processing unit and a Doppler-processing unit, which are not shown in the figure. The signal that has been outputted from the sending/receiving unit 12 is processed with the specified process either in this B-mode-processing unit or in this Doppler-processing unit. The B-mode-processing unit performs a band-pass filter (BPF)-process to the echo signal that has been sent from the send/receiving unit 12. Subsequently, the B-mode-processing unit detects an envelope and performs a compression process on the detected data using a logarithmic conversion.

The Doppler-processing unit generates information on blood flows by means of a pulse Doppler method or a continuous-wave Doppler method. The pulse Doppler method is used for the measurement of the velocity of tissues and blood flows located in the specified site. The continuous-wave Doppler method is used for the measurement of high-speed blood flows.

The Doppler processing unit performs phase detection on the receiving signal within a sample volume that has the specified size for the signal sent from the sending/receiving unit 12. The Doppler-processing unit extracts a Doppler-shift-frequency component by performing this phase detection. Furthermore, the Doppler-processing unit performs a fast Fourier transform (FFT) to the Doppler-shift-frequency component that has been extracted, and generates a Doppler frequency distribution that shows the blood-flow velocity within the sample volume of the same size.

Additionally, the Doppler-processing unit performs phase detection on the receiving signal on a sample line for blood-flow observation for the signals sent from the sending/receiving unit 12. The Doppler-processing unit extracts a Doppler-shift-frequency component by performing this phase detection, and generates the Doppler-frequency-component that shows the blood-flow velocity on the sample line by further performing an FFT process.

The image-processing unit 14 performs image processing for the signal sent from the signal-processing unit 13. For example, once the signal is sent as a signal column for scanning from the signal-processing unit 13, the image-processing unit 14 converts it into an image-signal column that is equivalent to the specified video format via DSC (digital scan converter).

Additionally, once the volume data is acquired by performing a volume scan for this image-signal column, the image-processing unit 14 performs volume rendering for said volume data. Subsequently, the image-processing unit 14 will generate three-dimensional image data by performing the volume rendering for the volume data.

Furthermore, in addition to generating the three-dimensional image data, the image-processing unit 14 generates arbitrary tomographic image data (MPR image data) by performing an MPR process (Multi Planar Reconstruction).

The acquisition-control unit 15 performs control to cause each unit in the image-acquisition unit 10 to perform a process as described above. In particular, the acquisition-control unit 15 receives an instruction from the display-processing unit 80 and relays the conditions for image acquisition to each unit in the image-acquisition unit 10. These include, for example, controls for the strength of the pulse sent by the sending/receiving unit 12, a frequency, a frame rate, the operating conditions for the ultrasonic probe, a delay control, and a gain at the time of receiving the reflected wave from the subject. The acquisition-control unit 15 also collects information on body tissues using an electrocardiograph 2 in synchronization with the heartbeats of the subject. In the case of synchronizing the electrocardiogram of the electrocardiograph 2, for example, the acquisition-control unit 15 receives a trigger signal from the electrocardiograph 2 when the electrocardiograph 2 acquires an electrocardiographic wave pattern and detects an R-wave. Subsequently, the acquisition-control unit 15 collects images obtained when the specified time has passed at a rate of one frame per heartbeat through a plurality of heartbeats.

As described above, the acquisition-control unit 15 controls each unit under the given conditions. At least one part of such conditions corresponds to the information on the examination conditions included in the supplemental information. Then, this information on the examination conditions is collected by the acquisition-control unit 15. The acquisition-control unit 15 sends the collected information on the examination conditions to the supplemental-information-acquisition unit 20. Furthermore, the acquisition-control unit 15 sends this collected information on the examination conditions to the video-data-generating unit 40.

The supplemental-information-acquisition unit 20 acquires the abovementioned information on the examination conditions and other supplemental information. This information on the examination conditions includes at least one of the following pieces of information. Examples of the information on the examination conditions include the conditions for reproducing images based on the image data, or information used as a reference for diagnosis using the ultrasonic images (e.g. a display magnification, a sweep speed, or the like, shown in a display unit 82 at the time of acquiring the images). Additionally, the information inputted by an operation unit 81 may also be included in the information on the examination conditions. Examples of other supplemental information include personal information such as information on patients, which should be protected.

Additionally, the supplemental-information-acquisition unit 20 links the supplemental information, including the information on the examination conditions, to a frame of the image data (unit for an image shown on a display screen) that existed at the same time as the time of acquiring the relevant information on the examination conditions. This is performed so that the superposition unit 70 described below can superpose the supplemental information in response to the changes over time of the ultrasonic images. The instructions for linking this information on the examination conditions with each frame are given to the supplemental-information-acquisition unit 20 from the record-control unit 50 after the record-control unit 50 described below receives the instructions for recording the video data of the ultrasonic images.

For example, one method for such linkage adds the time information of when the image-acquisition unit 10 collected the body-tissues information, to the frame of image data generated by the relevant body-tissues information. Additionally, the time information obtained when the acquisition-control unit 15 collects the information on the examination conditions is added to the information on the examination conditions. Furthermore, the supplemental-information-acquisition unit 20, based on both time information, identifies the information on the examination conditions that has been collected at the same time as the acquiring of the body-tissues information constituting each frame of the image data. Then, the supplemental-information-acquisition unit 20 links to each frame the information on the examination conditions identified in this way. The added time information is acquired by a timing means or the like provided in the ultrasonic imaging apparatus 1 and not shown in the figure. Alternatively, the information used for the linkage may be the information of the elapsed time starting from the time the image-acquisition unit 10 starts collecting the information on the body tissues.

Examples of the information items that can be linked with each frame of the image data as described above include synchronization information from an electrocardiogram, or data such as a display magnification and a sweep speed. Once the information on the examination conditions, including these items, is organized and linked with the corresponding relevant frame, the supplemental-information-acquisition unit 20 sends this information on the examination conditions to the supplemental-information-coding unit 60. Subsequently, once the record-control unit 50 described below receives the instruction for recording the video data related to the ultrasonic images, the superposition unit 70 superposes onto the image data the information on the examination conditions. A unit of superposing the information on the examination conditions and the image data will be described below. Next, FIG. 2 is a schematic view illustrating a superposing area of supplemental information in an ultrasonic imaging apparatus 1 according to the present embodiment.

As shown in FIG. 2, the screen-data-processing unit 30 stores a format and screen data, which are used for displaying the image display area other than ultrasonic image display area 201 within the display screen 200. This display screen 200 is comprised of video data. This video data is configured to include the relevant screen data, as well as data used for displaying the ultrasonic image display area 201 (i.e. image data showing the internal tissues of the subject that has been received by the image-processing unit 14).

Examples of data included in this screen data include data representing characters such as a logos, figures, or marks shown in area 203a, that do not change over time (corresponding to an example of “characters, figures, and marks, that do not change over time” in the present invention). The examples of data also include the data representing characters, figures, and graphs shown in a changeable area 203b/203c, which does change over time, and background information shown in the image display area 202.

Additionally, the screen-data-processing unit 30 performs data processing so that the synchronization information from the electrocardiogram 2 shown in the lower section of the display screen 200 in FIG. 2, as well as the supplemental information related to the ultrasonic images shown in the area 203a-203c, can be displayed at the time of displaying the ultrasonic images. Examples of this supplemental information include the patient ID, name, and examination date of a subject. The screen data that is processed and recorded in the screen-data-processing unit 30 is sent to the video-data-generating unit 40 upon the request of the video-data-generating unit 40.

The video-data-generating unit 40 receives the image data from the image-acquisition unit 10, and receives the screen data from the screen-data-processing unit 30. Subsequently, the video-data-generating unit 40 generates the video data that is used for displaying as an image on the display unit 82 by combining these acquired data. The generated video data is sent to the display-processing unit 80 and the record-control unit 50.

If the instruction is given by a user for recording the generated video data, the record-control unit 50 will acquire the generated video data from the video-data-generating unit 40 according to the instruction. Additionally, the record-control unit 50 sends the acquired video data to the superposition unit 70. The relevant user-instruction is given, for example, by the operation unit 81 provided in the display-processing unit 80.

The supplemental-information-coding unit 60 is configured to comprise an encrypting unit 61 and an error-correcting code-addition unit 62. This supplemental-information-coding unit 60 performs coding for the supplemental information. In other words, once the supplemental-information-coding unit 60 receives the supplemental information from the supplemental-information-acquisition unit 20, the encrypting unit 61 performs coding for the supplemental information, including personal information, to be protected so as to be undecipherable to observers of the ultrasonic images. Alternatively, the encrypting unit 61 performs invisualization, or encryption. Examples of this encryption include random number selection. The invisualization means that it prevents the viewers of the ultrasonic images from recognizing the personal information to be protected when the recorded ultrasonic images are reproduced and viewed.

The error-correcting code-addition unit 62 provided in the supplemental-information-coding unit 60 adds an error-correcting code, such as Reed Solomon (RS), to the supplemental information. This is performed for enhancing the resistance to image deterioration caused by zooming, varied resolution, and noninvertible (lossy) compression in a record-reproducing system. The supplemental-information-coding unit 60 sends the relevant supplemental information to the superposition unit 70 after necessary coding is performed for the supplemental information.

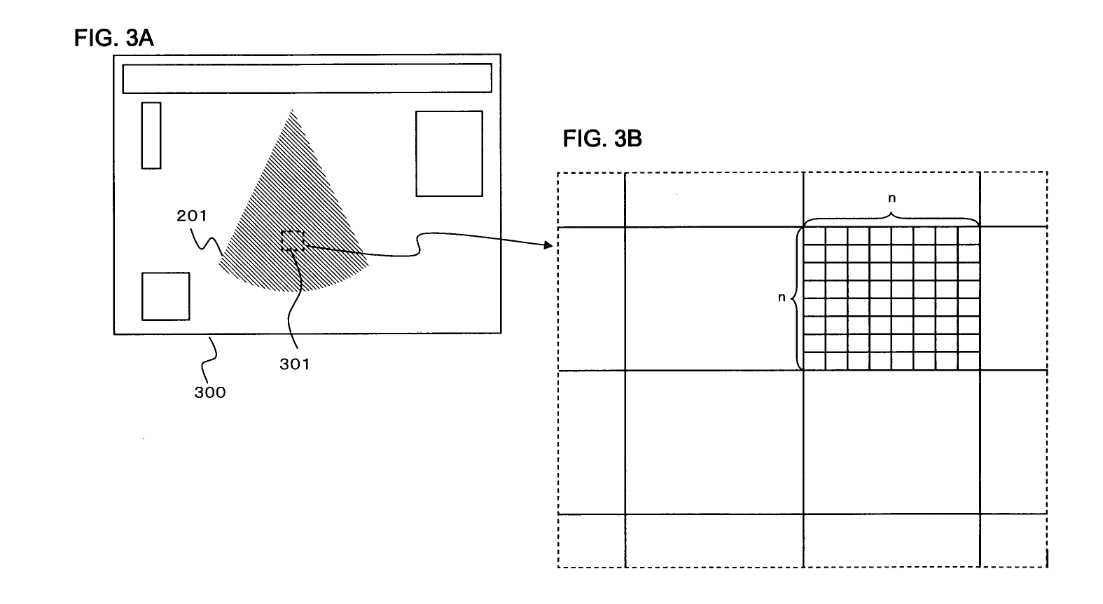

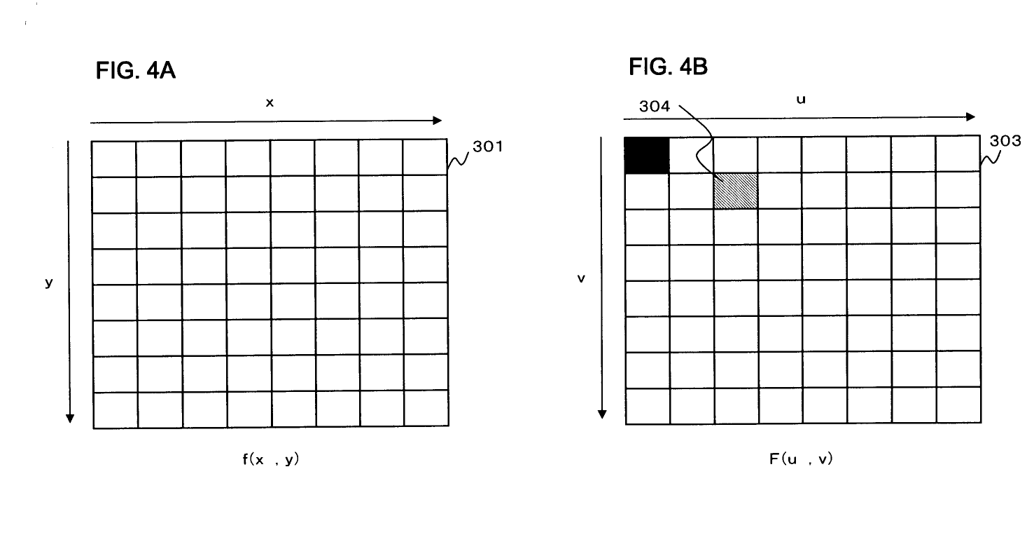

The superposition unit 70 acquires the video data from the record-control unit 50. The superposition unit 70 also acquires the supplemental information, including the information on the examination conditions, from the supplemental-information-coding unit 60. Upon acquisition of the video data and the supplemental information, the superposition unit 70 superposes data showing the specified area of the video data and the supplemental information. In order to perform a process of the superposition unit 70 featuring the present embodiment, the superposition unit 70 performs, for example, a discrete cosine transform (DCT) as an example of an image-data-transforming method. The superposition unit in the present embodiment will be described below with reference to FIG. 3 and FIG. 4.

The superposition unit 70 performs the following processes in order to superpose onto the image data the supplemental information, including the information on the examination conditions. First, the superposition unit 70 sequentially performs an orthogonal transform, such as discrete cosine transformation on the video data (at least on the image data) and decomposition on a spatial frequency.

This decomposition operation means that the video data is decomposed from low-frequency components to high-frequency components of the spatial frequency. As a method of the orthogonal transform, a Walsh-Hadamard transform (WHT), a discrete Fourier transform (DFT), a discrete sine transform (DST), a Haar transform, a Slant transform, or a Karhunen Loeve transform (KLT) may be used.

As described above, the superposition unit 70 superposes the supplemental information that has been coded by the supplemental-information-coding unit 60 onto the image data that has been performed with the decomposition to the spatial frequency by the orthogonal transformation. In the present embodiment, this orthogonal transformation is a discrete cosine transformation, and this coding uses, for example, random number selection.

As described above, the information on the examination conditions included in the supplemental information has information that has been linked with every frame. The frames are linked by the frame constituting the image data that shows the ultrasonic images (unit for an image shown on a display screen), or with every given number of frames (in the case of performing data compression by using the correlation relationship between frames). The superposition unit 70 superposes the frames related to the information on the examination conditions based on the relevant information.

Next, the superposition unit configured to superpose the screen data and the supplemental information will be described with reference to FIG. 3A/B and FIG. 4A/B. FIG. 3A is a schematic view of a display screen 300 illustrating a part of each frame of the image data. FIG. 3B is a schematic partial enlarged view illustrating the conditions after the ultrasonic image display area 201 of the display screen 300 is divided to a screen block 301. FIG. 4A is a schematic view illustrating a part of the conditions before an image block 301 of an image data frame undergoes orthogonal transformation. FIG. 4B is a schematic view illustrating the conditions of the process of superposing the supplemental information by performing an orthogonal transformation (e.g. a discrete cosine transformation) on an image block 301 of image data and decomposing to a spatial frequency in the superposition unit 70.

The display screen 300 shown in FIG. 3A illustrates each frame or specified-interval frame of the image data. In this display screen 300, the superposition unit 70 divides the ultrasonic image display area 201 into at least an image block 301 with n×n pixels. Additionally, the superposition unit 70, for example, performs a discrete cosine transformation on the divided image block 301 shown as f (x, y) in FIG. 4A. In this way, the superposition unit 70 transforms the image block 301 into the image block 303 shown as F (u, v) in FIG. 4B.

FIG. 4B illustrates a DC component with a spatial frequency of u=v=0. The higher the values of u and v, the higher the frequency the component has. Generally, due to this transformation, the image data focuses on the low-frequency component of the spatial frequency. This transformation is generally used, for example, for data compression. Consequently, the image block 301 is sequentially decomposed from a low-frequency component to a high-frequency component.

In this way, the superposition unit 70 transforms the specified area in every frame of the image data, with the result that the image block 301 is decomposed and large pixel value is focused on the low-frequency component. If the large pixel value is focused on the low-frequency component, free space will be created in the high-frequency component. The superposition unit 70 allocates the supplemental information into said free space data (i.e. the specified spatial frequency pixel 304 in the image block 303).

The method by which the superposition unit 70 allocates the supplemental information in the specified spatial frequency pixel 304 is performed, for example, by replacing the only value of the spatial frequency pixel 304 after the bit value 1 of the supplemental information is put as a positive given value and 0 is put as a negative given value.

At this stage, the location of the spatial frequency pixel in which the superposition unit 70 allocates, and the positive and negative given values, are determined according to the resistance to image deterioration caused by record-reproducing as well as the degree of encryption. At this stage, the location data obtained at the time when the superposition unit 70 allocates the supplemental information may be combined with the video data. Additionally, the superposition unit 70 may compress the video data by quantizing the image data that has undergone the discrete cosine transformation. However, it is the free space in the high-frequency component after the allocation of the supplemental information that is quantized.

In this way, the superposition unit 70 performs the discrete cosine transformation on the image data, and superposes the supplemental information linked with each frame or the given number of frames. Upon the completion of the superposition, the superposition unit 70 performs an inverse transformation on the video data superposed with the supplemental information in order to match the format method for recording in the recording device 3. This inverse transformation is performed in a method in response to the transformation method used for the superposition. In the present embodiment, since the superposition unit 70 performs the discrete cosine transformation on the image data, the superposition unit 70 performs an inverse discrete cosine transformation (IDCT) on the video data herein. It should be noted that if other orthogonal transformations are performed in the superposition unit 70, the inverse transformation of said transformation method will be performed.

Additionally, the superposition unit 70 sends the relevant video data to the video-format unit 90a after performing the inverse discrete cosine transformation on the video data in which the image data and supplemental information have been superposed.

The video-format unit 90a formats the video data that will be outputted to the recording device 3 in the form corresponding to the recording device 3. The format method of the video-format unit 90a is based on analog general standards such as the NTSC method, DV method digital VCR standards, and transmission system of uncompressed serial DVI method interface, and the like.

The video-format unit 90b receives the video data that has been outputted from the recording device 3, and formats it in response to the display-processing unit 80. Generally, the formatting method of the video-format unit 90b is a method that is to be an inverse transformation to the video-format unit 90a. Once the video data is formatted, the video-format unit 90b sends it to the decoding unit 100.

The decoding unit 100 receives the formatted video data from the video-format unit 90b, and transforms the video data again in the method corresponding to the transformation method conducted in the superposition unit 70. In the present embodiment, the superposition unit 70 performs a discrete cosine transformation on the video data herein, since the superposition unit 70 performs the discrete cosine transformation on the image data. It should be noted that if other orthogonal transformations are performed in the superposition unit 70, the decoding unit 100 will transform the video data in the transformation method again.

In other words, in the case that the discrete cosine transformation is performed on the video data for the superposition unit 70 to superpose the supplemental information, the decoding unit 100 divides the video data of each frame constituting the ultrasonic images into image blocks and performs a decomposition procedure sequentially by means of the discrete cosine transformation. This discrete cosine transformation is performed in order to extract the supplemental information superposed in the image data.

The supplemental-information-extraction unit 110 performs an extraction of the supplemental information from the image block that the discrete cosine transformation has been performed upon by the decoding unit 100. This extraction is performed by the supplemental-information-extraction unit 110, for example, based on the location information obtained when the superposition unit 70 allocates the supplemental information in the spatial frequency of each image block. Once the supplemental-information-extraction unit 110 extracts the supplemental information including the information on the examination conditions, the supplemental-information-extraction unit 110 sends the video data to the decoding unit 100. In the video data sent by this supplemental-information-extraction unit 110, each frame or frames of a specified interval are linked with the supplemental information including the information on the examination conditions.

The decoding unit 100 acquires the supplemental information that is linked with this video data. Once the decoding unit 100 acquires the extracted supplemental information, the inverse discrete cosine transformation is performed so that the video data that the discrete cosine transform has been performed upon can be reproduced. At this stage, the decoding unit 100 further performs an inverse quantization of the image data in cases where the superposition unit 70 has performed a quantization of the image data. The video data that has been decoded by performing the inverse discrete cosine transformation is sent by the decoding unit 100 to the display-processing unit 80 together with the supplemental information.

The display-processing unit 80 is configured to comprise a display unit 82 and an operation unit 81, and receives the video data from the video-data-generating unit 40 or the decoding unit 100 to display the video data, including the ultrasonic images. A display-control unit 83 completely controls the display unit 82, the operation unit 81, and each of the other units in the display-processing unit 80.

When the display-processing unit 80 receives the video data from the video-data-generating unit 40, in some cases, the display-control unit 82 will acquire the synchronization information on the electrocardiogram from the electrocardiograph 2 and display the image based on the video data and the electrocardiographic synchronization information. Meanwhile, when the display-processing unit 80 receives the video data from the decoding unit 100, this video data includes the supplemental information including the information on the examination conditions.

Accordingly, the display-control unit 82 displays the image based on the supplemental information, image data, and screen data in response to the display request made by the user. The display-processing unit 80 displays, for example, the electrocardiographic wave 204 or the like in FIG. 2 obtained from the information on the examination conditions included in the supplemental information.

At this stage, when the display-processing unit 80 receives the video data from the decoding unit 100, the display-processing unit 80 may constantly display on the image the supplemental information including the information on the examination conditions that has been encrypted and included in the relevant video data.

Additionally, with the ultrasonic images displayed on the display unit 82, using the operation unit 81, the users (operators) can specify on the ultrasonic images the location where the ultrasonic images should be acquired (probe mark and body mark). At this time, the display-processing unit 80 identifies and displays the location of the ultrasonic images that has been specified based on the information on the examination conditions included in the supplemental information.

Operation

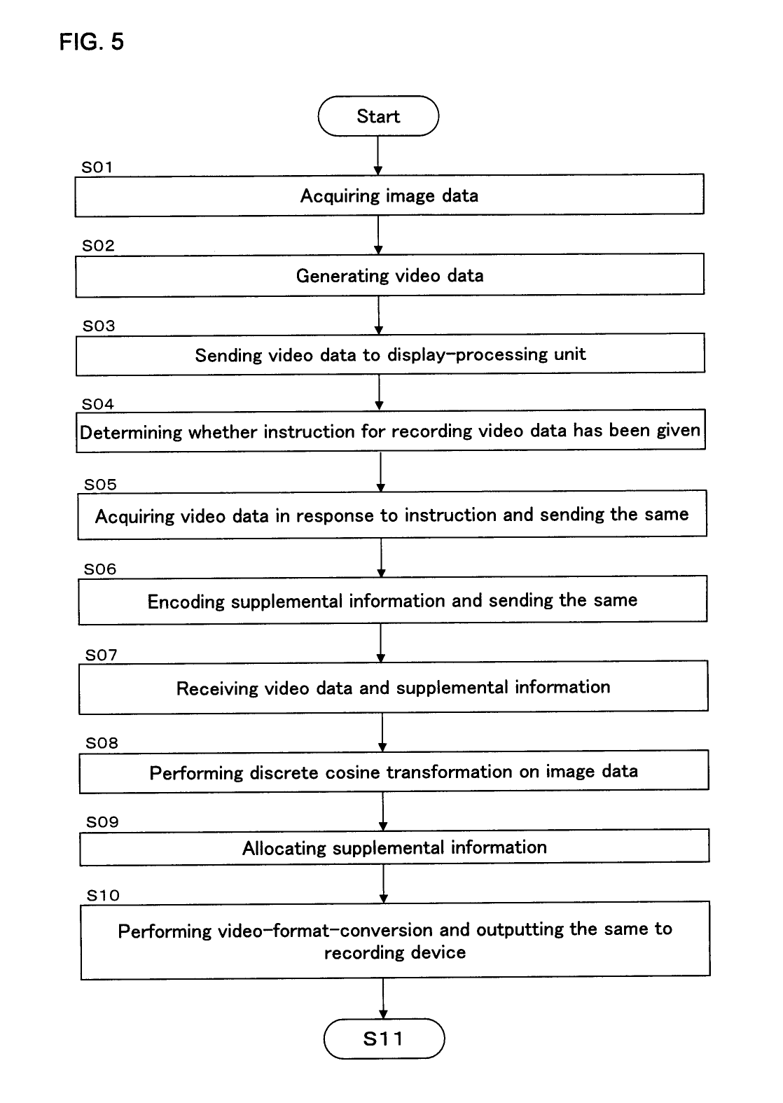

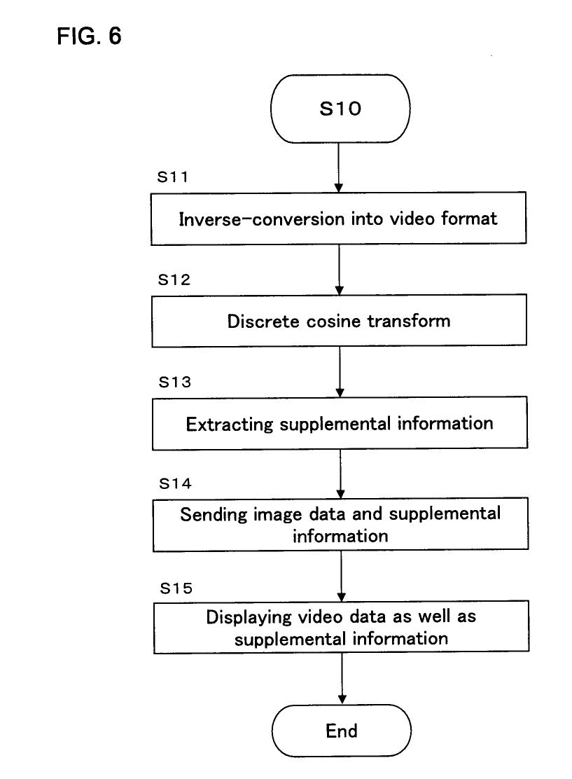

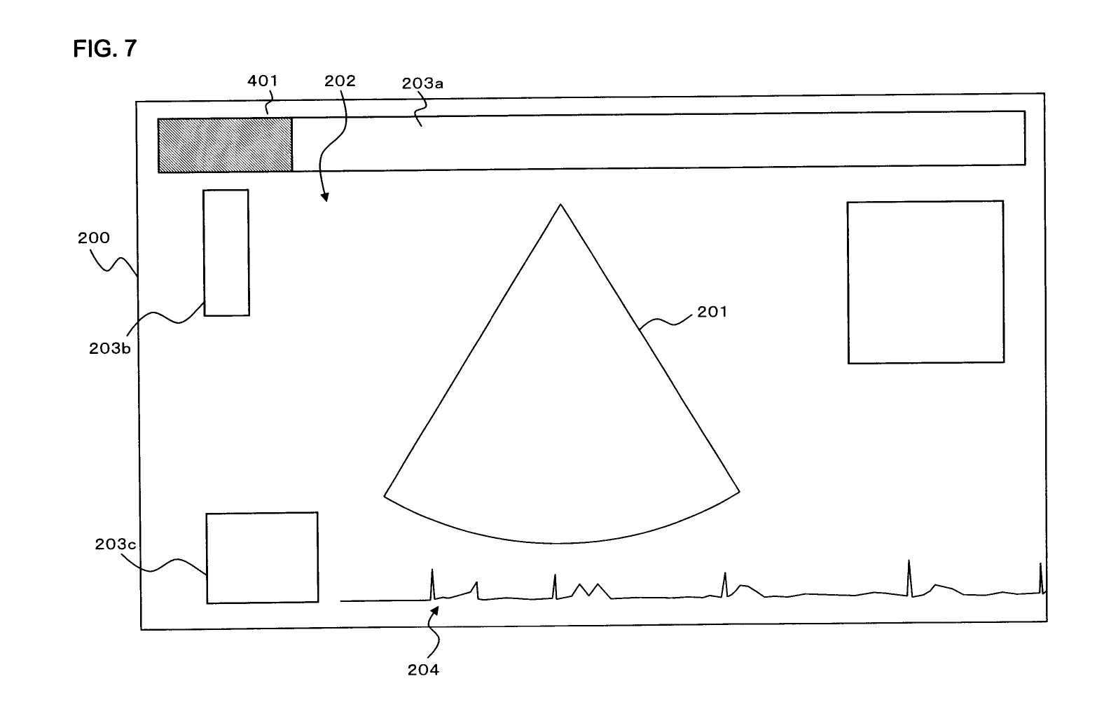

Next, the operations of an ultrasonic imaging apparatus 1 according to the first embodiment of the present invention will be described with reference to FIG. 7. FIG. 5 and FIG. 6 are flow charts illustrating a series of operations performed by the ultrasonic imaging apparatus 1.

Step 1

First, using an ultrasonic probe or the like, in order to acquire image data using an ultrasonic imaging apparatus 1, a user (e.g. a physician) performs work for collecting the information on body tissues for each frame constituting image data. Once the user performs the relevant work, an acquisition-control unit 15 controls each unit in an image-acquisition unit 10 in accordance with the relevant work. In other words, the acquisition-control unit 15 receives a reflected wave from the inside of the subject by means of an ultrasonic probe and converts the relevant reflected wave into an electric signal. Then, according to control by the acquisition control unit 15, a sending/receiving unit 12 receives the relevant converted signal, a signal-processing unit 13 performs signal processing, and an image-processing unit 14 performs a control for generating the image data. Additionally, when the acquisition-control unit 15 synchronizes with an electrocardiograph 2 at the time of acquiring the image data, the acquisition-control unit 15 performs a control for collecting the information on the body tissues using the electrocardiograph in synchronization with the heartbeats of the subject.

Step 2

Once the image data is generated by the image-acquisition unit 10, the acquisition-control unit 15 performs a control for sending the video data to a video-data-generating unit 40. Upon receipt of the image data, the video-data-generating unit 40 requests screen data from a screen-data-processing unit 30 and obtains the screen data. The video-data-generating unit 40 combines the relevant image data and the screen data to generate the video data.

Step 3

Once the video-data-generating unit 40 generates the video data, the generated video data is sent to a display-processing unit 80. The display-processing unit 80 controls the display-control unit 82 to cause the display unit 82 to display the ultrasonic images. Upon the request of the user, the display unit 82 displays the display screen based on the video data reflected by the information on the examination conditions. At this stage, if the display-processing unit 80 acquires the synchronization information from the electrocardiograph 2, it will display the synchronization information on the electrocardiogram 2 synchronized with the relevant video data that is being displayed on the display unit 82 in real time. If there is other information on the examination conditions acquired from the image-acquisition unit 10, the display-processing unit 80 will display other received data by reflecting them on the video data.

Step 4

Once the video data is generated, a record-control unit 50 provided in the ultrasonic imaging apparatus 1 displays it so that the user can judge whether the relevant generated video data is recorded on the display unit 82 in the display-processing unit 80. At this stage, by means of an operation unit 81 (e.g. inputting unit), the user can select whether the recording is to be performed or not, in order to give an instruction for recording to the record-control unit 50. Additionally, the record-control unit 50 determines whether the instruction for recording the video data has been given by this user or not.

Step 5

When the record-control unit 50 determines that the instruction for recording the video data has been given, the record-control unit 50 sends the video data acquired from the video-data-generating unit 40 in response to the instruction for recording to a superposition unit 70. Additionally, if the record-control unit 50 determines that the instruction for recording the video data has not been given, the video data is not sent to the superposition unit 70.

Step 6

Once the video data is sent to the superposition unit 70, the record-control unit 50 controls a supplemental-information-acquisition unit 20 so that the supplemental information, including the information on the examination conditions, is sent to a supplemental-information-coding unit 60. Once the supplemental information is sent to the supplemental-information-coding unit 60, the supplemental-information-coding unit 60 encodes the supplemental information in an encryption unit 61 and in an error-correcting code-addition unit 62, and then sends it to the superposition unit 70. Additionally, before the supplemental information is sent from the supplemental-information-acquisition unit 20 to the supplemental-information-coding unit 60, the record-control unit 50 causes the supplemental-information-coding unit 20 to link the information on the examination conditions with each frame constituting the image data based on time information and the like.

Step 7

Once the video data and the supplemental information are received, the superposition unit 70 links the information on the examination conditions with each frame of the image data based on the time information linked with the information on the examination conditions included in the supplemental information. That is, based on the time information linked with each frame or specified-interval frame of the image data in the relevant video data, the corresponding information on the examination conditions is linked with each of the relevant frame.

Step 8

For the video data received from the record-control unit 50 as described above, the superposition unit 70 divides it into image blocks with specified numbers of pixels, and performs a discrete cosine transformation on each image block (e.g. image block 301 or the like).

Step 9

After performing the discrete cosine transformation on the relevant image block 301, the superposition unit 70 determines a spatial frequency pixel that is suitable for allocating the supplemental information. The determination of the pixel location conducted by this superposition unit 70 is as described above. Once the pixel location has been determined, the superposition unit 70 allocates to the relevant determined spatial frequency pixel the supplemental information, including the information on the examination conditions, that has been linked with each frame or specified-interval frame of the image data in Step 8. Once the supplemental information is superposed onto the image data, the superposition unit 70 performs the inverse discrete cosine transformation on the video data. The superposition unit 70 sends the video data including the relevant image data upon which the inverse discrete cosine transformation has been performed to the video-format-conversion unit 90a.

Step 10

Once the video data is received from the superposition unit 70, the video-format-conversion unit 90a converts it into the data format for the recording device 3. The video-format-conversion unit 90a outputs this converted video data to the recording device 3.

Step 11

When the user makes a request to reproduce the video data that has been recorded, the recording device 3 outputs the video data. After receiving the video data that has been outputted from the recording device 3, the video-format-conversion unit 90b performs an inverse conversion into a data format for the ultrasonic image acquisition device 1. Additionally, the video-data-format-conversion unit 90b sends the video data on which the inverse conversion has been performed to a decoding unit 100.

Step 12

Once the converted video data is received from the video-format-conversion unit 90b, the decoding unit 100 divides it into blocks with the specified number of pixels and performs a discrete cosine transformation as performed in the superposition unit 70.

Step 13

The supplemental-information-extraction unit 110 extracts the supplemental information from the image block of the image data on which the discrete cosine transformation has been performed by the decoding unit 100.

Step 14

The decoding unit 100 links the supplemental information extracted by the supplemental-information-extraction unit 100 with the image data. The decoding unit 100 performs the inverse discrete cosine transformation on the video data after linking the supplemental information with the image data. The decoding unit 100 sends the video data to the display-processing unit 80 after performing the inverse discrete cosine transformation on the video data.

Step 15

Once the display-processing unit 80 receives the video data from the decoding unit 100, the display-control unit 82 causes the display unit 82 to display the video data, including the image data related to the ultrasonic image data, based on the relevant video data. If the video data is linked with the supplemental information (e.g. the synchronization information on the electrocardiogram), the display-control unit 83 will cause the display-control unit 82 to display the supplemental information, such as the information on the examination conditions, that has been linked with each frame of the image data.

Actions/Effects

Actions and effects of the ultrasonic imaging apparatus 1 according to the above present embodiment will be described.

The ultrasonic imaging apparatus 1 according to the present embodiment is configured to superpose on the image data the supplemental information including the information on the examination conditions and then output it to the recording device 3 when recording the video data in the recording device. This supplemental information is necessary to perform a re-measurement that requires reproducing the recorded video data. In addition, when reproducing the video data that the has been recorded in the ultrasonic imaging apparatus 1, it is configured to comprise a transformation unit and an inverse transformation unit, which correspond to the same transformation method as performed at the time of recording the video data. Further, the ultrasonic imaging apparatus 1 is configured to comprise a supplemental-information-extraction unit that extracts the supplemental information from the transformed video data.

Accordingly, since the superposition unit 70 records the supplemental information by superposing it onto the image data itself, when the video data recorded in the ultrasonic imaging apparatus 1 is reproduced to be displayed, the information on the examination conditions that changes over time can be linked with the ultrasonic images. In addition, since the superposition unit 70 records the supplemental information by superposing it onto the image data itself, the addition, transmission, and recording of the supplemental information can be performed without dependence on any specific data format.

In other words, when the video data recorded in a specified data format is reproduced in the device that has another data format, since the supplemental information is superposed onto the image data to be reproduced itself, the information on the examination conditions that is necessary to reproduce and re-measure the video data can be read, even between different data formats.

Consequently, even in cases where the video data is recorded in the recordable media in the ultrasound imaging apparatus 1, the user can mutually use the video data even if the format is different between the devices for the recording and the devices for the reproducing.

Furthermore, since the ultrasonic imaging apparatus 1 is configured so that the supplemental information is superposed onto the image data by the superposition unit 70, the supplemental information is displayed on ultrasonic diagnostic images that change over time, and displayed in the indistinctive form at the time of the observation of the image, so there is few possibility of interference in the observation of the ultrasonic diagnostic.

Additionally, the ultrasonic imaging apparatus 1 according to the present embodiment comprises an encrypting unit 61 provided in the supplemental-information-coding unit 60, and the encrypting unit 61 performs an encryption or an obfuscation process so that the viewers of the ultrasonic images cannot easily recognize the personal information included in the supplemental information. Accordingly, the user manages the confidentiality of the personal information of the subject that should be protected, and therefore the personal information can be properly protected. For example, the encrypted personal information (e.g. “aaa” for a patient name) can be decoded only by the specified person (e.g. physician), who can protect the confidentiality of the personal information of the subject.

Furthermore, the ultrasonic imaging apparatus 1 according to the present embodiment comprises the error-correcting code-addition unit 62 provided in the supplemental-information-coding unit 60. This error-correcting code-addition unit 62 uses the coding methods in which correcting codes such as the Reed Solomon error-correcting code are added, which can lead to enhanced resistance to data deterioration.

### Second Embodiment

Configuration

The configuration of an ultrasonic imaging apparatus according to the second embodiment of the present invention will be described. It should be noted that the codes for each unit described below is based on FIG. 1.

In an ultrasonic imaging apparatus according to the second embodiment, operations in the supplemental-information-coding unit 60, the superposition unit 70, the decoding unit 100, and the supplemental-information-extraction unit 110, are different from the operations in the ultrasonic imaging apparatus 1 according to the first embodiment described above. However, the other parts in the ultrasonic imaging apparatus according to the second embodiment are the same as the ultrasonic imaging apparatus 1 according to the first embodiment. Only the different operations in each unit for the ultrasonic imaging apparatus according to the second embodiment will be described below.

The supplemental-information-coding unit 60 according to the second embodiment is configured to comprise an error-correcting code-addition unit. The error-correcting code-addition unit encrypts the supplemental information. This error-correcting code-addition unit also performs an encryption by receiving the supplemental information sent from the supplemental-information-acquisition unit 20, just like the supplemental-information-coding unit 60 in the first embodiment. Once the supplemental information is encrypted, the supplemental-information-coding unit 60 sends this supplemental information to the superposition unit 70. In this coding method, coding using a QR code such as a two-dimensional bar code is performed. It should be noted that the supplemental information used herein is the same data as the supplemental information described above.

The superposition unit 70 according to the second embodiment superposes the video data with the supplemental information after acquiring the video data from the record-control unit 50 and acquiring the supplemental information from the supplemental-information-coding unit 60. The superposition unit 70 according to the present embodiment is different from the superposition unit 70 according to the first embodiment in that the supplemental information that has been coded is superposed by drawing it onto the screen data. At this stage, the superposition unit 70 adds the location information related to the superposition area (superposition location) of the supplemental information to the video data (or screen data) so that the reproducing device for the video data can extract the superposed supplemental information later.

The superposition unit 70 according to the second embodiment first draws and superposes the supplemental information onto the screen data in the video data, in order to superpose the supplemental information onto the screen data. In this embodiment, different from the first embodiment, when displaying the video data recorded in the reproducing device, the superposed supplemental information may be perceived as a character or a pattern as long as the superposed supplemental information does not interfere with the diagnosis. For example, the superposition unit 70 according to the second embodiment may superpose a logo and a mark onto the display area 401 for displaying characters and marks that do not change over time, as shown in FIG. 7.

The superposition unit 70 superposes the supplemental information onto the logo and mark, and therefore the user can perceive this logo and mark itself when reproducing the video data in the reproducing device. However, the user can perceive the logo and mark but will not recognize the personal information as long as the superposition area for this supplemental information is not decoded. In this way, the superposition unit 70 according to the second embodiment properly manages personal information.

Receiving the formatted video data from the video-format-conversion unit 90b, the decoding unit 100 according to the second embodiment cause the supplemental-information-extraction unit 110 to extract the data related to the area where the supplemental information in the screen data is superposed onto by drawing. The supplemental-information-extraction unit 110 sends the extracted data to the decoding unit 100. The decoding unit 100 decodes the coded supplemental information received from the supplemental-information-extraction unit 110. At this stage, the supplemental information is extracted by the decoding unit 100 on the basis of the location information related to the superposition area that has been added to the video data or the screen data.

Decoding the supplemental information, the decoding unit 100 links with the image data the information on the examination conditions that has been linked with each frame or specified-interval frame of the image data. Linking the information on the examination conditions with the image data, the decoding unit 100 sends the video data related to the image data to the display-processing unit 80.

Operation

One example of operations for the ultrasonic imaging apparatus 1 according to the second embodiment as described above will be described. Since the operational order up to the superposition process of the superposition unit 70 (i.e. the operational order up to the process where the record-control unit 50 sends the video data to the superposition unit 70 and the supplemental-information-coding unit 60 sends the supplemental information to the superposition unit 70) is the same as the processes of the ultrasonic imaging apparatus 1 according to the first embodiment, a description thereof is omitted herein.

Receiving the video data and the supplemental information, the superposition unit 70 draws and superposes the supplemental information onto the data showing the area for displaying characters and marks (e.g. the area for displaying characters and marks shown in FIG. 7) among the screen data. The information on the superposition area of the supplemental information is added to the screen data in order to extract the superposed supplemental information later.

Since the process of outputting the data to the recording device 3 by converting the video data in the video-format-conversion unit 90a, as well as the process of sending the data to the recording device 3 by converting the video data in the video format 90b according to the second embodiment, are the same as the process in the video format 90a/90b, a description thereof is omitted herein.

Receiving the video data from the video-format unit 90b, the decoding unit 100 identifies the screen data from the video data. Then, the decoding unit 100 sends the identified screen to the supplemental-information-extraction unit 110.

After receiving the screen data from the decoding unit 100, the supplemental-information-extraction unit 110 extracts the relevant superposition area based on the information on the superposition area of the supplemental information that has been added in advance. The supplemental-information-extraction unit 110 sends the extracted data to the decoding unit 100. The decoding unit 100 decodes the data showing the extracted superposition area among the screen data. Additionally, the decoding unit 100 links the supplemental information with the image data. Furthermore, the decoding unit 100 sends the video data including the image data to the display-processing unit 80.

Once the supplemental information is extracted, the supplemental information is linked with the image data based on the information that has been linked with each frame or specified-interval frame, and then the video data is sent to the display-processing unit 80.

Once the display-processing unit 80 receives the video data from the decoding unit 100, the display-control unit 83 causes the display unit 82 to display ultrasonic images related to the video data and the video including other information, based on the relevant data. If the supplemental information, such as the information on the examination conditions (e.g. synchronization information on an electrocardiogram), has been linked with the video data, the display-control unit 82 causes the display unit 82 to display the supplemental information, such as the information on the examination conditions, linked with each frame of the image data as the relevant other information.

Actions/Effects

The actions and effects of the ultrasonic imaging apparatus 1 according to the present embodiment described above will be described.

The ultrasonic imaging apparatus 1 according to the present embodiment is configured, when recording the video data in the recording device, to superpose onto the image data the supplemental information including the information on the examination conditions and then output to the recording device 3. This supplemental information is necessary to perform a re-measurement with reproducing the recorded video data. Additionally, when reproducing the video data that has been recorded in the ultrasonic imaging apparatus 1, a transformation unit and an inverse transformation unit are provided, which correspond to the same transformation methods as performed at the time of recording the video data. Furthermore, the ultrasonic imaging apparatus 1 is configured to comprise a supplemental-information-extraction unit that extracts the supplemental information from the transformed video data.

As described above, the superposition unit 70 records the supplemental information by superposing it onto the image data itself. Therefore, when the video data recorded in the ultrasonic imaging apparatus 1 is reproduced to display, the information on the examination conditions that changes over time can be linked with the ultrasonic images. In addition, since the superposition unit 70 records the supplemental information by superposing it onto the image data itself, the addition, transmission, and recording of the supplemental information can be performed without dependence on any specific data format. Therefore, the recorded video data is mutually used even between different data formats.

Additionally, the ultrasonic imaging apparatus 1 according to the present embodiment is configured to draw and superpose the supplemental information onto the data showing the area for displaying characters and marks among the screen data. Therefore, even if the superposition unit 70 superposes the supplemental information, there will be no influence on the image data showing the image of internal tissues of the subject to be examined among the video data. Additionally, since the superposition unit 70 draws the supplemental information within the area for displaying characters and marks, it is not necessary for the supplemental information to be encrypted. Furthermore, even though the supplemental information is displayed at the time of the observation conducted by the user, it can be displayed in an indistinctive form, which results in few possibility of interference in the observation of the relevant image.

Furthermore, the supplemental-information-coding unit 60 provided in the ultrasonic imaging apparatus 1 according to the present embodiment comprises an error-correcting code-addition unit performed by a two-dimensional bar code such as a QR code, which can lead to enhanced resistance to data deterioration.

### MODIFIED EXAMPLE

Next, modified examples of the abovementioned embodiment will be described.

### Modified Example 1

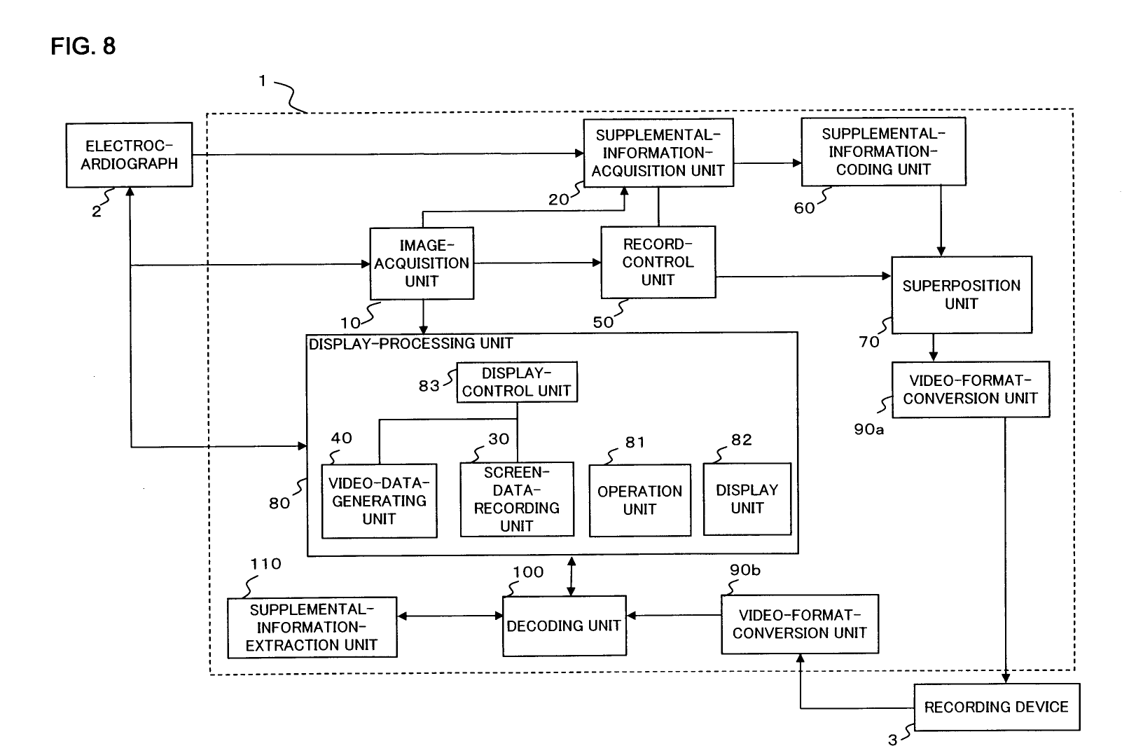

The ultrasonic imaging apparatus according to the first embodiment is configured to send the video data to the superposition unit 70 through the video-data-generating unit 40. The present invention is not limited to this embodiment. For example, the ultrasonic imaging apparatus may be configured as shown in FIG. 8.

In other words, the ultrasonic imaging apparatus of this modified example 1 as shown in FIG. 8 may be configured so that the screen-data-processing unit 30 and the video-data-processing unit 40 are included in the display-processing unit 80. In addition, the superposition unit 70 may be configured to acquire the image data directly from the image-acquisition unit 10. In this case, all the data that is outputted from the ultrasonic imaging apparatus 1 to the recording device 3 is to be image data superposed with the supplemental information. Additionally, in this case, the display-processing unit 80 also performs a process of generating the video data by combining the image data and the screen data when acquiring the image data and displaying the ultrasonic images. Even in this configuration, the same effects as the ultrasonic imaging apparatus 1 according to the abovementioned first embodiment can be achieved.

### Modified Example 2

The ultrasonic imaging apparatus according to the first embodiment is configured to perform an orthogonal transformation such as a discrete cosine transformation on the video data to superpose the supplemental information, and also to further perform an inverse discrete cosine transformation on the relevant video data for outputting to the recording device 3. However, the present invention is not limited to this embodiment. For example, in the modified example 2, the data is compressed to record using a discrete cosine transformation such as the MPEG (Moving Picture Experts Group) method, which is a method for recording video data. In this configuration, it is not necessary to perform the inverse discrete cosine transformation when outputting the video data from the ultrasonic imaging apparatus 1 to the recording device 3. In addition, after the video data is received from the recording device 3 to the ultrasonic imaging apparatus 1, it is not necessary to perform the discrete cosine transformation again. For this reason, a plurality of transformation work and inverse transformation work performed in the ultrasonic imaging apparatus 1 can be omitted, and the configuration in the ultrasonic imaging apparatus 1 can be simplified. Even in this configuration, the same effects as the ultrasonic imaging apparatus 1 according to the above-mentioned first embodiment can be achieved.

### Modified Example 3

In the ultrasonic imaging apparatus 1 according to the second embodiment, after the video data is generated by the video-data-generating unit 40, the video-data-generating unit 40 sends the video data to the superposition unit 70. The superposition unit 70 superposes the supplemental information onto the screen data out of the video data received from the video-data-generating unit 40. The present invention is not limited to this embodiment. For example, in the modified example 3 of the present invention, the superposition unit 70 acquires the screen data from the screen-data-processing unit 30. Then, after superposing the screen data and the supplemental information from the superposition unit 70, these data are sent to the video-data-generating unit 40. The video-data-generating unit 40 may be configured to generate the video data after receiving the screen data superposed with the supplemental information. Even in this configuration, the same effects as the ultrasonic imaging apparatus 1 according to the above-mentioned second embodiment can be achieved.

### Modified Example 4

In the ultrasonic imaging apparatus 1 according to the second embodiment, the supplemental information that has been coded in the supplemental-information-coding unit 60 is drawn and superposed in the superposition unit 70 onto the data showing the area for displaying characters and marks among the screen data. The present invention is not limited to this embodiment. For example, in the modified example 4 of the present invention, the supplemental information is randomized in the supplemental-information-coding unit 60. The superposition unit 70 superposes this randomized supplemental information onto the screen data. The ultrasonic imaging apparatus 1 may encrypt the supplemental information in this way. The superposition unit 70 superposes this encrypted supplemental information onto the screen data, which leads to invisualization for the supplemental information, even when displaying the ultrasonic imaging that has this screen data reproduced and recorded in the reproducing device. In that case, the supplemental information may be added with an error-correcting code. Even in this configuration, the same effects as the ultrasonic imaging apparatus 1 according to the abovementioned second embodiment can be achieved.

### Modified Example 5

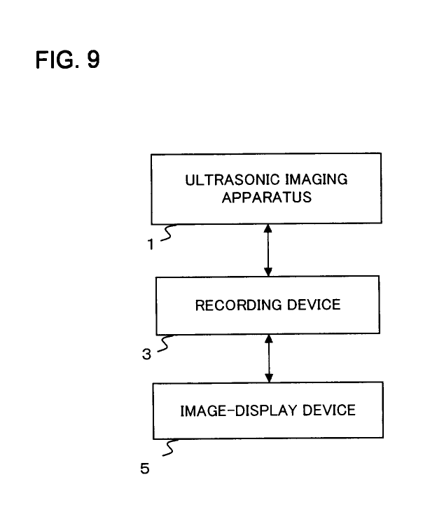

The operations of the ultrasonic imaging apparatus 1 according to the first embodiment and the second embodiment include the processes of superposing the supplemental information onto the video data generated in the ultrasonic imaging apparatus, converting the relevant video data into a video format, and outputting it to the recording device 3. Secondly, the ultrasonic imaging apparatus 1 includes the processes of receiving the video data recorded in the recordable media from the recording device 3, decoding the image data or the screen data, extracting the supplemental information, and reproducing the video data linked with the supplemental information. The present invention is not limited to these embodiments. For example, the video data recorded by the ultrasonic imaging apparatus 1 can be configured to be reproduced in other devices as shown in FIG. 9. In other words, in the modified example 3 of the present invention as shown in FIG. 9, an image-display device 5 comprising a unit for decoding the image data that has been coded after receiving the video data or the screen data from the recording device 3, a unit for extracting the supplemental information, and a unit for reproducing the video data while linking the supplemental information with the video data, may be configured to reproduce the video data.

It should be noted that the ultrasonic imaging apparatus is explained in the above description, the present invention is also applicable to other modalities such as an X-ray CT apparatus or an MRI apparatus.

What is claimed is:

1. An ultrasonic imaging apparatus, comprising:
an acquisition unit configured to radiate ultrasound to a subject to be examined, and to acquire image data that shows the internal condition of said subject to be examined;
a video-data-generating unit configured to generate video data to be displayed on a display screen, based on said image data and screen data to display an image display area different from an area in which the image based on said image data is displayed; and
a superposition unit configured to superpose information on the examination conditions onto said image data;
wherein said video data including the image data superposed with said information on the examination conditions is outputted to a recording device.

2. An ultrasonic imaging apparatus, comprising:
an acquisition unit configured to radiate ultrasound to a subject to be examined, and to acquire image data that shows the internal condition of said subject to be examined;
a video-data-generating unit configured to generate video data to be displayed on a display screen, based on said image data and screen data to display an image display area different from an area in which the image based on said image data is displayed; and
a superposition unit configured to superpose information on the examination conditions onto data to display characters, figures, marks or patterns that do not change over time out of said screen data;
wherein said video data, including the screen data superposed with said information on the examination conditions, is outputted to a recording device.

3. The ultrasonic imaging apparatus according to claim 1, comprising:
an extraction unit configured to receive video data superposed with said information on the examination conditions from said recording device, and to extract said information on the examination conditions from said image data in said video data.

4. The ultrasonic imaging apparatus according to claim 2, comprising:
an extraction unit configured to receive video data superposed with said information on the examination conditions from said recording device, and to extract said information on the examination conditions from said screen data in said video data.

5. The ultrasonic imaging apparatus according to claim 3, further comprising:
a coding unit configured to code said information on the examination conditions superposed with said video data so as to be undecipherable to observers of said video data.

6. The ultrasonic imaging apparatus according to claim 4, further comprising:
a coding unit configured to code said information on the examination conditions superposed with said video data so as to be indecipherable to observers of said video data.

7. The ultrasonic imaging apparatus according to claim 5,
wherein said coding unit encrypts said information on the examination conditions for invisualization, and
said superposition unit superposes said information on the examination conditions encrypted by said coding unit.

8. The ultrasonic imaging apparatus according to claim 6,
wherein said coding unit encrypts said information on the examination conditions for invisualization, and
said superposition unit superposes said information on the examination conditions encrypted by said coding unit.

9. The ultrasonic imaging apparatus according to claim 5,
wherein said coding unit receives said video data and said information on the examination conditions, and transforms said image data in said video data into spatial frequency area data, and
said superposition unit superposes by allocating said information on the examination conditions in free area of said transformed spatial frequency area data.

10. The ultrasonic imaging apparatus according to claim 6,
wherein said coding unit receives said video data and said information on the examination conditions, and transforms data to display characters, figures, marks, or patterns that do not change over time out of said screen data in said video data into spatial frequency area data, and
said superposition unit superposes by allocating said information on the examination conditions in free area of said transformed spatial frequency area data.

11. The ultrasonic imaging apparatus according to claim 7,
wherein said extraction unit receives from said recording device video data that is configured to have said screen data and said image data superposed with said information on the examination conditions, extracts said encrypted information on the examination conditions from said image data in said video data, and decodes said extracted information on the examination conditions.

12. The ultrasonic imaging apparatus according to claim 8,
wherein said extraction unit receives from said recording device video data that is configured to have said screen data and said image data superposed with said information on the examination conditions, extracts said encrypted information on the examination conditions from said image data in said video data, and decodes said extracted information on the examination conditions.

13. The ultrasonic imaging apparatus according to claim 1, wherein said information on the examination conditions includes at least one of either the operating conditions on said acquisition unit at the time of acquiring said image data, or biological information on a subject to be examined showing the conditions of tissues of the subject to be examined that is different from said image data, and further comprising an addition unit configured to add error-correcting codes to said information on the examination conditions and said video data.

14. The ultrasonic imaging apparatus according to claim 2,
wherein said information on the examination conditions includes at least one of either the operating conditions on said acquisition unit at the time of acquiring said image data, or biological information on a subject to be examined showing the conditions of tissues of the subject to be examined that is different from said image data, and further comprising an addition unit configured to add error-correcting codes to said information on the examination conditions and said video data.

15. A viewer, comprising:
an extraction unit configured to receive video data via a recordable media in which video data including: image data showing the internal conditions of a subject to be examined and are superposed with information on the examination conditions; and screen data to display an image display area different from an area in which the image based on said image data is displayed, are recorded, and extracting the information on the examination conditions from said image data; and
a display unit configured to display video data that includes said image data and reflects said information on the examination conditions.

16. A viewer, comprising:
an extraction unit configured to receive screen data via a recordable media in which video data including image data showing the internal conditions of a subject to be examined, screen data including coding data to display characters, figures, marks, and patterns that do not change over time for displaying an image display area different from an area in which that image based on said image data is displayed, and the information on the examination conditions superposed with said coding data; and
extracting the information on the examination conditions from coding data in said screen data; and
a display unit configured to display video data that includes said image data and reflects said information on the examination conditions.

17. A method for acquiring ultrasonic images, causing an ultrasonic imaging apparatus to perform steps of:
acquiring information on a subject to be examined by radiating ultrasound to the subject to be examined, and acquiring image data showing the internal conditions of said subject to be examined based on the information of said subject to be examined;
generating video data that includes said image data and screen data to display an image display area different from an area in which the image based on said image data is displayed;
superposing onto said image data the information on the examination conditions that includes at least one of either the operating conditions at the time of acquiring said image data, or biological information on the subject to be examined showing the conditions of tissues of the subject to be examined that is different from said image data; and
outputting said video data, including said image data superposed with said information on the examination conditions, to a recording device.

18. A method for acquiring ultrasonic images, causing an ultrasonic imaging apparatus to perform steps of:
acquiring information on a subject to be examined by radiating ultrasound to the subject to be examined, and acquiring image data showing the internal conditions of said subject to be examined based on the information of said subject to be examined;
generating video data that includes said image data and screen data to display an image display area different from an area in which the image based on said image data is displayed;
superposing the information on the examination conditions that includes at least one of either the operating conditions of said ultrasonic imaging apparatus at the time of acquiring said image data, or biological information on a subject to be examined showing the conditions of tissues of the subject to be examined that is different from said image data, onto data to display characters, figures, marks, or patterns that do not change over time out of said screen data; and
outputting said video data including said image data superposed with said information on the examination conditions to a recording device.

19. A method for acquiring ultrasonic images according to claim 17, causing an ultrasonic imaging apparatus to perform steps of:
receiving said video data from said recording device and extracting said superposed information on the examination conditions from said image data in said video data; and
reproducing and displaying said video data that reflects said extracted information on the examination conditions.

20. A method for acquiring ultrasonic images according to claim 18, causing an ultrasonic imaging apparatus to perform steps of:
receiving said video data from said recording device and extracting said superposed information on the examination conditions from said screen data; and
reproducing and displaying said video data that reflects said extracted information on the examination conditions.

21. A medical imaging apparatus, comprising:
an acquisition unit configured to acquire information on the interior of a subject to be examined, and generating image data showing the internal conditions of said subject to be examined based on the information on the interior of said subject to be examined;
a video-data-generating unit configured to generate video data that includes said image data and screen data to display an image display area different from an area in which the image based on said image data is displayed; and
a superposition unit configured to superpose the information on the examination conditions that includes at least one of either the operating conditions on said acquisition unit at the time of acquiring said image data, or biological information on the subject to be examined showing the conditions of tissue of the subject to be examined that is different from said image data, onto said image data out of said video data;
wherein said video data including said image data superposed with said information on the examination conditions by said superposition unit is outputted to a recording device.

22. A medical imaging apparatus, comprising:
a generation unit configured to acquire the information on the interior of a subject to be examined and generating image data that shows the internal conditions of said subject to be examined;
a video-data-generating unit configured to generate video data that includes said image data and screen data to display an image display area different from an area in which the image based on said image data is displayed;
a superposition unit configured to superpose the information on the examination conditions that includes at least one of either the operating conditions on said acquisition unit at the time of acquiring said image data, or biological information on said subject to be examined showing the conditions of the tissues of the subject to be examined that is different from said image data, onto data to display characters, figures, marks and or patterns that do not change over time, out of said screen data in said video data; and
wherein said video data including said screen data superposed with said information on the examination conditions by said superposition unit is outputted to a recording device.
